(19) United States
(12) Patent Application Publication
(10) Pub. No.: US 2026/0265451 A1
(43) Pub. Date: Sep. 10, 2026

(54) LIQUID BIOPOLYMER, USE THEREOF, AND PREPARATION METHOD

(71) Applicant: LG CHEM, LTD., Seoul (KR)

(72) Inventors: Jae Hyung KIM, Daejeon (KR); Dong Gyun KANG, Daejeon (KR); Chul Woong KIM, Daejeon (KR); Young Hyun CHO, Daejeon (KR); Sung Joon OH, Daejeon (KR); Jeong Kyu LEE, Daejeon (KR); In Young HUH, Daejeon (KR)

(21) Appl. No.: 19/224,579

(22) Filed: May 30, 2025

Related U.S. Application Data
(62) Division of application No. 15/769,045, filed on Mar. 9, 2017, now abandoned.

(30) Foreign Application Priority Data
Mar. 28, 2016 (KR) 10-2016-0037218

(51) Int. Cl. C08G 63/06; C07K 14/21; C09D 167/04; C12P 7/625

(52) U.S. Cl. CPC C08G 63/06; C07K 14/21; C09D 167/04; C12P 7/625; C08G 2230/00

(57) ABSTRACT

A biopolymer, which exists in a liquid phase at room temperature, a use thereof, and a preparation method therefor are provided.

### CROSS-REFERENCE TO RELATED APPLICATION(S)

This application is a Divisional Application of U.S. patent application Ser. No. 15/769,045, filed on Apr. 17, 2018, which is a National Stage of International Application No. PCT/KR2017/002585, filed on Mar. 9, 2017, which claims the benefit of Korean Patent Application No. 10-2016-0037218 filed on Mar. 28, 2016, the contents of which are all hereby incorporated by reference herein in their entirety.

### INCORPORATION BY REFERENCE OF SEQUENCE LISTING PROVIDED AS A SEQUENCE LISTING XML FILE

The Sequence Listing associated with this application is provided electronically in XML file format and is hereby incorporated by reference in its entirety. The electronic XML file containing the Sequence Listing was created on May 30, 2025, is 45,000 bytes in size, and is titled 29137.02632SEQUENCELISTING.xml.

### TECHNICAL FIELD

The present invention provides a polyhydroxyalkanoate(PHA) biopolymer which is present in a liquid phase at room temperature, its use and a preparation method thereof.

### BACKGROUND ART

Biopolymers are polymeric plastics produced by using biomass as a raw material. They are collectively called not only plastics composed of biomass-based components only but also mixtures comprising petrochemical-based plastics. Biopolymers are environmentally friendly materials whose main components are plastics made from plants and microorganisms which can be easily decomposed and converted into a form that can be absorbed by living organisms.

Polyhydroxyalkanoate (PHA), a typical type of a biopolymer, is a natural polyester material that is accumulated in the microorganism for the storage of energy and reducing capability when the microorganism is abundant in carbon sources in the absence of elements required for growth such as nitrogen, oxygen, phosphorus, magnesium, etc. Since PHA exhibits biodegradability and biocompatibility while having properties similar to those of synthetic polymers derived from petroleum, it is recognized as a substitute for conventional synthetic plastics.

About 150 types of PHA monomers are known, and most of these monomers are 3-, 4-, 5- or 6-hydroxyalkanoate (HA). Representative PHA monomers that are actively studied are monomers having hydroxyl groups at carbon positions 3 and 4, such as 3-hydroxybutyrate (3HB), 4-hydroxybutyrate (4HB), 3-hydroxypropionate (3HP), and 3-hydroxyalkanoate having medium chain length (MCL) of 6 to 12 carbon atoms.

An enzyme that plays a key role in the synthesis of PHA in a microorganism is the PHA synthase, which synthesizes a polyester containing the corresponding monomer, using various hydroxyacyl-CoA as a substrate. In addition, since the PHA synthase has substrate specificity for various hydroxyacyl-CoA's, the monomer composition of the polymer is controlled by the PHA synthase. Therefore, in order to synthesize PHA, a metabolic pathway for synthesizing and providing various hydroxyacyl-CoA which can be used as a substrate of PHA synthase and a metabolic pathway for polymer synthesis using the substrate and PHA synthase are required.

### DETAILED DESCRIPTION OF THE INVENTION

### Technical Problem

Conventional PHA biopolymers are present in a solid state at room temperature. The present invention provides a biopolymer that exists in a liquid phase at room temperature, and further provides a biopolymer that not only is present in a liquid phase at room temperature but also exhibits biodegradability and adhesive properties, and thus can be utilized in various fields.

### DETAILED DESCRIPTION OF THE INVENTION

### Technical Problem

One embodiment provides a polyhydroxyalkanoate (PHA) biopolymer present in a liquid phase at room temperature.

Another embodiment provides a PHA biopolymer composition which is biodegradable or hydrophobic or has both biodegradability and hydrophobicity at the same time, comprising the biopolymer.

Still another embodiment provides a method for preparing a copolymer containing 4-hydroxybutyrate and 2-hydroxybutyrate as a repeating unit, comprising a step of culturing a microorganism which has a weakened or deficient activity of lactate dehydrogenase and contains a gene encoding an enzyme converting 2-hydroxyalkanoate into 2-hydroxyalkanoyl-CoA and converting 4-hydroxyalkanoate into 4-hydroxyalkanoyl-CoA and a gene encoding a polyhydroxyalkanoate synthase using 2-hydroxyalkanoyl-CoA and 4-hydroxyalkanoyl-CoA as a substrate.

Still another embodiment provides a microorganism which has a weakened or deficient activity of lactate dehydrogenase, contains a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate, and produces a copolymer containing 4-hydroxybutyrate and 2-hydroxybutyrate as a repeating unit.

A further embodiment provides a method for preparing a microorganism which produces a copolymer containing 4-hydroxybutyrate and 2-hydroxybutyrate as a repeating unit, comprising the steps of: deleting a gene encoding for lactate dehydrogenase and introducing a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate into a cell.

### Advantageous Effects

The present invention provides a PHA biopolymer that is present in a liquid phase at room temperature, and the biopolymer can be widely used as a raw material for biodegradable, biocompatible, and hydrophobic bioplastics in electronic, automobile, food, agricultural and medical fields. In particular, the liquid PHA polymer provided herein exhibits excellent adhesive properties, and thus can be applied to the entire chemical industry such as paints, color paints, coatings, polymers, fibers and adhesives. Further, it can be applied as a medical bio-adhesive since it does not dissolve in water and retains adhesive properties even in a wet state. For example, various medical applications such as tissue adhesives, hemostatic agents, supports for tissue engineering, drug delivery carriers, tissue fillers, wound healing, or prevention of adhesion between tissues are possible.

### BRIEF DESCRIPTION OF DRAWINGS

FIG. 1 shows the fabrication process and cleavage map of the pPs619C1310-CpPCT540 vector.

FIG. 2 shows the cleavage map of the pPs619C1249.18H-CPPCT540 vector.

FIG. 3 shows the result of gas chromatography analysis of the 4-hydroxybutyrate-2-hydroxybutyrate copolymer produced from recombinant cells.

FIG. 4 shows a photograph of a polymer comprising 4-hydroxybutyrate and 2-hydroxybutyrate at various molar ratios.

FIG. 5 shows the results of a differential scanning calorimetry (DSC) analysis of a polymer including 4-hydroxybutyrate and 2-hydroxybutyrate at various molar ratios. Endo represents an endothermic reaction, and exo represents an exothermic reaction.

### DETAILED DESCRIPTION OF THE EMBODIMENTS

In one embodiment, the present invention relates to a polyhydroxyalkanoate (PHA) biopolymer present in a liquid phase at room temperature.

A specific embodiment relates to a PHA biopolymer which exists in a liquid phase at room temperature and has biodegradability or hydrophobicity, or has both biodegradability and hydrophobicity simultaneously.

Another specific embodiment relates to a PHA biopolymer present in a liquid phase at room temperature, comprising 4-hydroxybutyrate and 2-hydroxybutyrate as a repeating unit.

Another specific embodiment relates to a biopolymer present in a liquid phase at room temperature, wherein the polymer comprises 4-hydroxybutyrate and 2-hydroxybutyrate as a repeating unit and 4-hydroxybutyrate and 2-hydroxybutyrate are contained in the polymer in a molar ratio of 30% or more, respectively.

Another specific embodiment relates to a biopolymer present in a liquid phase at room temperature, wherein the polymer includes 4-hydroxybutyrate and 2-hydroxybutyrate as a repeating unit and 4-hydroxybutyrate and 2-hydroxybutyrate are contained in the polymer in a molar ratio of 40% or more, respectively.

Another specific embodiment relates to a biopolymer present in a liquid phase at room temperature, wherein the polymer comprises 4-hydroxybutyrate and 2-hydroxybutyrate as a repeating unit and 4-hydroxybutyrate and 2-hydroxybutyrate are contained in the polymer in a molar ratio of 1:1.

Another embodiment relates to a biopolymer composition having biodegradability and hydrophobicity at the same time, comprising the biopolymer.

A specific embodiment relates to a biopolymer composition which can be adhered to a substrate selected from the group consisting of glass, metal, polymeric materials, hydrogels, wood, ceramics, cells, tissues, organs, and biomolecules.

Another specific embodiment relates to a biopolymer composition which can be used as a tissue adhesive, a tissue suture agent, an adhesion inhibitor, a hemostatic agent, a support for tissue engineering, wound dressing, a drug delivery carrier, a tissue filler, an environmentally friendly paint, an environmentally friendly oil color, a hair loss concealer additive, or a cosmetic additive.

Another embodiment relates to a method for preparing a copolymer containing 4-hydroxybutyrate and 2-hydroxybutyrate as a repeating unit.

Another embodiment relates to a method for preparing a copolymer containing 4-hydroxybutyrate and 2-hydroxybutyrate as a repeating unit, comprising a step of culturing a cell which has a weakened or deficient activity of lactate dehydrogenase and contains a gene encoding an enzyme converting 2-hydroxyalkanoate into 2-hydroxyalkanoyl-CoA and converting 4-hydroxyalkanoate into 4-hydroxyalkanoyl-CoA and a gene encoding a polyhydroxyalkanoate synthase using 2-hydroxyalkanoyl-CoA and 4-hydroxyalkanoyl-CoA as a substrate.

In another embodiment, the present invention relates to a microorganism producing a copolymer containing 4-hydroxybutyrate and 2-hydroxybutyrate as a repeating unit and a preparation method thereof.

A specific embodiment relates to a microorganism which has a weakened or deficient activity of lactate dehydrogenase, contains a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate, and produces a copolymer containing 4-hydroxybutyrate and 2-hydroxybutyrate as a repeating unit.

Another embodiment relates to a method for preparing a microorganism which produces a 4-hydroxybutyrate-2-hydroxybutyrate copolymer, comprising the steps of: deleting a gene encoding for lactate dehydrogenase and introducing a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate into a cell.

Hereinafter, the present invention will be described in more detail.

The present invention provides a PHA biopolymer present in a liquid phase at room temperature. Preferably the present invention provides a PHA biopolymer present in a liquid phase at room temperature and normal pressure.

Room temperature refers to a normal temperature that is not particularly heated or controlled, and may generally be in the temperature range of 15° C. to 30° C. or 20° C. to 25° C. Normal pressure refers to a normal atmospheric pressure which is not particularly pressurized or controlled, and may generally be a pressure range of about 900 to 1,100 hPa.

In one embodiment, the biopolymer has biodegradability. Biodegradability refers to a property that can be degraded in vivo.

In another embodiment, the biopolymer has hydrophobicity. Hydrophobicity refers to a property that is difficult to bind to water molecules.

In another embodiment, the biopolymer has both biodegradability and hydrophobicity at the same time.

The PHA polymer includes a polymer composed of various hydroxyalkanoate monomers without limitation, as long as it is present in a liquid phase at room temperature and normal pressure. For example, the hydroxyalkanoate monomer may be 2-, 3-, 4-, 5- or 6-hydroalkenoate.

In one embodiment, the term “copolymer containing 4-hydroxybutyrate and 2-hydroxybutyrate as a repeating unit” refers to PHA polymer which is a linear polyester containing a repeating unit obtained by polymerizing monomers 4-hydroxybutyrate and 2-hydroxybutyrate through ester bonds. At this time, there is no particular limitation on the order of polymerization of the respective monomers, and they can be repeated randomly. Examples thereof include a 4-hydroxybutyrate-2-hydroxybutyrate copolymer, or 2-hydroxybutyrate-4-hydroxybutyrate copolymer.

In one embodiment of the present invention, polyalkanoate copolymer macromolecules including 4-hydroxybutyrate and 2-hydroxybutyrate in various molar ratios were prepared and analyzed for physical properties. In differential scanning calorimetry (DSC) analysis, while crystallization was observed in a homopolymer of 4-hydroxybutyrate or 2-hydroxybutyrate, it was confirmed that a copolymer of 4-hydroxybutyrate and 2-hydroxybutyrate was an amorphous polymer in which the crystallization and the melting temperature (Tm) were not observed. It was also confirmed for the first time that copolymers of 4-hydroxybutyrate and 2-hydroxybutyrate exhibited adhesive properties. In particular, it was confirmed that, when the molar ratios of 4-hydroxybutyrate and 2-hydroxybutyrate monomers were 30% or more, respectively, the copolymer exhibited proper liquid phase properties, hydrophobicity and adhesive property for an adhesive. In addition, when the molar ratios of the 4-hydroxybutyrate and the 2-hydroxybutyrate monomer are 40% or more, the copolymer can exhibit proper liquid phase properties, hydrophobicity and adhesive property for an adhesive. In addition, when the molar ratio of the 4-hydroxybutyrate and the 2-hydroxybutyrate monomer is 1:1, the copolymer can exhibit proper liquid phase properties, hydrophobicity and adhesive property for an adhesive.

Thus, 4-hydroxybutyrate and 2-hydroxybutyrate in the copolymer may be provided in a molar ratio of 30:70 to 70:30, or 40:60 to 60:40, or 50:50, and the copolymer may be present in a liquid phase at room temperature and normal pressure. Also, within the above range, the copolymer of the present invention can exhibit adhesive properties. Further, within the above range, since the copolymer of the present invention exhibits biocompatibility, hydrophobicity and adhesiveness as well as being present in a liquid phase, it can be used as an adhesive for adhering or fixing glass, metal, polymeric materials, hydrogels, wood, ceramics or biological materials. In addition, the polymer of the present invention can be used as a medical bioadhesive since it does not dissolve in water and retains its adhesive property even in a wet state.

Accordingly, the present invention also provides a biopolymer composition having both biodegradability and hydrophobicity at the same time, comprising a biopolymer present in a liquid phase at room temperature.

For example, the biopolymer composition may be a solvent type, a water-soluble type, or a non-solvent type, and may be used in an amount of 0.01 to 100 μg/cm² based on the substrate, but is not limited thereto. In addition, the method of using the composition is in accordance with a conventional method of using a biopolymer, and a typical method may be a coating method.

The biopolymer composition of the present invention can be adhered to a variety of substrates such as inanimate surfaces or biological samples. For example, the composition can be adhered to, but are not limited to, substrates selected from the group consisting of glass, metal, polymeric materials, hydrogels, wood, ceramics, cells, tissues, organs, and biomolecules. Examples of biomolecules may include, but are not limited to, nucleic acids, amino acids, peptides, proteins, lipids, carbohydrates, enzymes, hormones, growth factors or ligands.

Accordingly, the biopolymer composition of the present invention can be used not only in the chemical industry such as paints, color paints, coatings, polymers, films, adhesive sheets and fibers, but also in a various application such as the automobile industry, electric and electronic industries, cosmetics, medicine and pharmacy.

For example, the biopolymer composition can be used as a tissue adhesive, a tissue suture agent, an adhesion inhibitor, a hemostatic agent, a support for tissue engineering, wound dressing, a drug delivery carrier, a tissue filler, an environmentally friendly paint, an environmentally friendly oil color, a hair loss concealer additive, or a cosmetic additive.

As a specific example, the biopolymer composition can be used in various fields such as skin, blood vessels, digestive system, cranial nerve, plastic surgery, orthopedic surgery, etc. instead of cyanoacrylic adhesives or fibrin-based adhesives which are currently used in the market. For example, the biopolymer composition can replace surgical sutures, can be used for occluding unnecessary blood vessels and for hemostasis and suture of soft tissues such as facial tissues and cartilage and hard tissues such as bones and teeth, and can be applied for a household medicine.

More specifically, the biopolymer composition can be applied to the inner and outer surfaces of the human body as a bioadhesive, and can be locally applied to, for example, the outer surface of the human body, such as skin, or the surface of an internal organ exposed during a surgical procedure. In addition, the biopolymer composition of the present invention can be used to adhere damaged portions of tissue, to seal air/fluid leaks in tissue, to adhere medical devices to tissues, or to fill defective portions of tissue. The term “biological tissue” is not particularly limited and includes, for example, skin, bone, nerve, axon, cartilage, blood vessel, cornea, muscle, muscle fascia, brain, prostate, breast, endometrium, lung, spleen, small intestine, liver, testis, ovary, cervix, rectum, stomach, lymph node, bone marrow, and kidney.

In addition, the biopolymer composition can be used for wound healing. For example, it can be used as a dressing applied to a wound.

In addition, the biopolymer composition can be used for skin suture. That is, it can be used topically to suture the wound, replacing the suture thread. In addition, the biopolymer composition of the present invention can be applied to hernia repair, for example, can be used for coating surface of meshes used for hernia repair.

The biopolymer composition can also be used to suture and prevent leakage of tubular structures such as blood vessels. In addition, the biopolymer composition of the present invention can also be used for hemostasis.

In addition, the biopolymer composition may be used as an adhesion inhibitor. Adhesion occurs at all surgical sites and is a phenomenon where other tissues stick around the wound around the surgical site. Adhesion occurs in about 97% of cases after surgery, and in particular, 5-7% of them cause serious problems. In order to prevent such adhesion, wound is minimized during surgery or anti-inflammatory drugs may be used. In addition, TPA (tissue plasminogen activator) is activated to prevent the formation of fibrin, or physical barriers such as crystalline solution, polymer solution, and solid membrane are used. However, these methods can be toxic in vivo and may exhibit other side effects. The biopolymer composition of the present invention can be applied to the tissue exposed after surgery to prevent adhesion that occurs between the tissue and the surrounding tissue. For example, it can be used as an agent for preventing organ adhesion, especially as an intestinal adhesion inhibitor.

The biopolymer composition may also be used as a support for tissue engineering. Tissue engineering technology refers to a technique of culturing a cell isolated from a patient's tissue on a support to prepare a cell-support complex and transplanting the complex into the body. Tissue engineering technique is applied to a regeneration of almost all organ of human body, including artificial skin, artificial bone, artificial cartilage, artificial cornea, artificial blood vessel, artificial muscles, and the like. Since the biopolymer composition of the present invention can be adhered to various biomolecules, it can be used as a support for tissue engineering. Further, the biopolymer composition can be used as a medical material such as a cosmetic material, a wound covering material, and a dental matrix.

In addition, the biopolymer composition can be used for ophthalmic adhesions such as a treatment of perforation, fissure, or incision, corneal transplantation, and artificial corneal insertion; dental adhesions such as retainer appliances, dental bridges, crown attachment, shaking tooth fixation, broken tooth treatment, and filling material fixation; surgical treatment such as vascular inosculation, cell tissue inosculation, artificial material transplantation, wound closure; orthopedic treatments such as treatment of bones, ligaments, tendons, meniscus and muscle and artificial material transplantation; or a drug delivery carrier or the like.

The term “enzyme that converts 2-hydroxyalkanoate into 2-hydroxyalkanoyl-CoA and converts 4-hydroxyalkanoate into 4-hydroxyalkanoyl-CoA” refers to an enzyme capable of producing 2-hydroxyalkanoyl-CoA and 4-hydroxyalkanoyl-CoA by dissociating CoA from a CoA donor and transferring it to 2-hydroxyalkanoate and 4-hydroxyalkanoate, respectively. Examples of the CoA donor include acetyl-CoA or acyl-CoA (e.g., propionyl-CoA).

In one embodiment, the enzyme may be a propionyl-CoA transferase. In addition, the gene of the enzyme may be derived from Clostridium propioni cum.

For example, a gene encoding for the enzyme that converts 2-hydroxyalkanoate, 3-hydroxyalkanoate and 4-hydroxyalkanoate into 2-hydroxyalkanoyl-CoA, 3-hydroxyalkanoyl-CoA and 4-hydroxyalkanoyl-CoA, respectively, may have a nucleotide sequence selected from the group consisting of the following:
(a) a nucleotide sequence of SEQ ID NO: 1;
(b) a nucleotide sequence of SEQ ID NO: 1 comprising A1200G mutation;
(c) a nucleotide sequence of SEQ ID NO: 1 comprising T78C, T669C, A1125G and T1158C mutations;
(d) a nucleotide sequence of SEQ ID NO: 1 comprising A1200G mutation and a mutation resulting in Gly335Asp mutation in the amino acid sequence corresponding to SEQ ID NO: 1;
(e) a nucleotide sequence of SEQ ID NO: 1 comprising A1200G mutation and a mutation resulting in Ala243Thr mutation in the amino acid sequence corresponding to SEQ ID NO: 1;
(f) a nucleotide sequence of SEQ ID NO: 1 comprising T669C, A1125G and T1158C mutations and a mutation resulting in Asp65Gly mutation in the amino acid sequence corresponding to SEQ ID NO: 1;
(g) a nucleotide sequence of SEQ ID NO: 1 comprising A1200G mutation and a mutation resulting in Asp257Asn mutation in the amino acid sequence corresponding to SEQ ID NO: 1;
(h) a nucleotide sequence of SEQ ID NO: 1 comprising T669C, A1125G and T1158C mutations and a mutation resulting in Asp65Asn mutation in the amino acid sequence corresponding to SEQ ID NO: 1;
(i) a nucleotide sequence of SEQ ID NO: 1 comprising T669C, A1125G and T1158C mutations and a mutation resulting in Thr199Ile mutation in the amino acid sequence corresponding to SEQ ID NO: 1; and
(j) a nucleotide sequence of SEQ ID NO: 1 comprising T78C, T669C, A1125G and T1158C mutations and a mutation resulting in Val93Ala mutation in the amino acid sequence corresponding to SEQ ID NO: 1.

The term “PHA synthase using 2-hydroxyalkanoyl-CoA and 4-hydroxyalkanoyl-CoA as a substrate” refers to an enzyme capable of synthesizing a copolymer comprising 4-hydroxybutyrate and 2-hydroxybutyrate as a repeating unit using 2-hydroxyalkanoyl-CoA and 4-hydroxyalkanoyl-CoA as a substrate.

For example, the enzyme may be PHA synthase (phaC) derived from Pseudomonas sp.6-19.

For example, the PHA synthase may consist of a nucleotide sequence corresponding to an amino acid sequence of SEQ ID NO: 4 or an amino acid sequence of SEQ ID NO: 4 comprising at least one mutation selected from the group consisting of L18H, V24A, K91R, M128V, E130D, N246S, S325T, S477R, S477H, S477F, S477Y, S477G, Q481M, Q481K, Q481R and A527S.

In another specific example, the PHA synthase may consist of a nucleotide sequence corresponding to an amino acid sequence of SEQ ID NO: 4 comprising a mutation selected from the group consisting of:
(i) S325T and Q481M;
(ii) E130D, S325T and Q481M;
(iii) E130D, S325T, S477R and Q481M;
(iv) E130D, S477F and Q481K; and
(v) L18H, V24A, K91R, M128V, E130D, N246S, S325T, S477G, Q481K and A527S.

Such enzymes may include additional mutations within the scope of not totally altering the activity of the molecule. For example, amino acid exchanges in proteins and peptides that do not generally alter the activity of the molecule are known in the art. For example, commonly occurring exchanges are the amino acid residue exchanges such as Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu and Asp/Gly, but are not limited thereto. In some cases, the protein may be modified by phosphorylation, sulfation, acrylation, glycosylate ion, methylation, farnesylation, etc. In addition, an enzyme protein having increased structural stability against heat, pH and the like or increased protein activity due to mutations or modification of the amino acid sequence may be included.

In addition, a gene encoding the enzyme may include a nucleic acid molecule comprising a functionally equivalent codon, a codon encoding the same amino acid (by codon degeneracy), or a codon encoding a biologically equivalent amino acid. The nucleic acid molecule may be isolated or prepared using standard molecular biology techniques, for example, a chemical synthesis method or a recombinant method, or a commercially available nucleic acid molecule may be used.

The term “lactate dehydrogenase” refers to an enzyme that catalyzes the reversible conversion between pyruvic acid and lactate, and the enzyme plays an essential role in the lactate synthesis pathway. In one embodiment, the gene encoding the lactate dehydrogenase may be IdhA.

The present invention is characterized in that lactate dehydrogenase, which is involved in the production of lactate during the metabolism of the host cell, is weakened or deficient compared with the intrinsic regulatory activity in order to produce a lactate-free copolymer. The intrinsic regulatory activity means the active state of the enzyme that the host cell has in its natural state, and may mean, for example, the activity of lactate synthesis naturally occurring in Escherichia coli.

The deletion of the lactate dehydrogenase activity can be carried out by genetic manipulation which deletes or substitutes part or all of the gene encoding the enzyme or inserts a specific mutation sequence into the nucleotide sequence of the gene. In addition, the activity of lactate dehydrogenase may be weakened by modifying the nucleotide sequence of the expression regulatory sequence of the gene such as the promoter region or the 5′-UTR region of the gene to weaken the expression of the enzyme, or by introducing a mutation at the region of open reading frame to weaken the activity of the enzyme. The introduction of such a mutation can be accomplished by any method known in the art, for example, homologous recombination, or lambda red recombination system.

The microorganisms provided herein comprise a gene encoding an enzyme converting 2-hydroxyalkanoate into 2-hydroxyalkanoyl-CoA and converting 4-hydroxyalkanoate into 4-hydroxyalkanoyl-CoA and a gene encoding PHA synthase using 2-hydroxyalkanoyl-CoA and 4-hydroxyalkanoyl-CoA as a substrate, and the genes may have been introduced into the microorganisms by a genetic recombination method.

For example, the microorganism may be the one obtained by transforming a microorganism with a recombinant vector comprising a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate, or by genetically engineering a microorganism to insert the genes on its chromosome.

In addition, the cells may be the one which has already one gene among a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate. In this case, the other gene may be transformed into the cell by a recombinant vector or inserted into a chromosome of the cell by a genetic manipulation.

For example, the microorganism may be the one obtained by transforming a cell comprising a gene encoding a PHA synthase using 2-hydroxyalkanoyl-CoA and 4-hydroxyalkanoyl-CoA as a substrate with a gene encoding an enzyme that converts 2-hydroxyalkanoate into 2-hydroxyalkanoyl-CoA and converts 4-hydroxyalkanoate into 4-hydroxyalkanoyl-CoA.

In another example, the microorganism may be the one obtained by transforming a cell comprising a gene encoding an enzyme that converts 2-hydroxyalkanoate into 2-hydroxyalkanoyl-CoA and converts 4-hydroxyalkanoate into 4-hydroxyalkanoyl-CoA with a gene encoding a PHA synthase using 2-hydroxyalkanoyl-CoA and 4-hydroxyalkanoyl-CoA as a substrate.

A process for preparing a microorganism which produces 4-hydroxybutyrate-2-hydroxybutyrate copolymer by a genetic recombination method or for producing 4-hydroxybutyrate-2-hydroxybutyrate copolymer using the microorganism may include the following steps.

First, at least one of a gene encoding an enzyme that converts 2-hydroxyalkanoate into 2-hydroxyalkanoyl-CoA and converts 4-hydroxyalkanoate into 4-hydroxyalkanoyl-CoA and a gene encoding a PHA synthase using 2-hydroxyalkanoyl-CoA and 4-hydroxyalkanoyl-CoA as a substrate is inserted into a vector to produce a recombinant vector. The two genes can be inserted into separate vectors or inserted into a single vector.

The term “vector” refers to a gene construct comprising an essential regulatory element operably linked to enable expression of a gene insert encoding a desired protein in a cell of an individual, and can be a means to introduce a nucleic acid sequence encoding a desired protein into a host cell. Various types of vectors such as a plasmid, a virus vector, a bacteriophage vector, cosmid vector, and a YAC (Yeast Artificial Chromosome) vector can be used. Recombinant vectors include cloning vectors and expression vectors. A cloning vector is a replicon that contains a replication origin, for example, a replication origin of a plasmid, phage, or cosmid, and another DNA fragment attached and the attached DNA fragment can be replicated. Expression vectors have been developed to be used to synthesize proteins.

In the present invention, the vector is not particularly limited as long as it functions to express and produce the desired enzyme gene in various host cells such as prokaryotic cells or eukaryotic cells. However, a vector that allows the gene introduced into the vector to be transferred and irreversibly fused into the genome of the host cell and gene expression to be stably maintained for a long period of time in a cell is preferable.

Such vectors include transcriptional and translational expression regulatory sequences that allow a gene to be expressed in a selected host. Expression regulatory sequences may include promoters for conducting transcription, any operator sequences for regulating such transcription, sequences encoding suitable mRNA ribosome binding sites, and/or sequences regulating the termination of transcription and translation. For example, regulatory sequences suitable for prokaryotes may include a promoter, optionally an operator sequence and/or a ribosome binding site. Regulatory sequences suitable for eukaryotic cells may include promoters, terminators and/or polyadenylation signals. An initiation codon and a termination codon are generally considered to be part of a nucleic acid sequence encoding a desired protein, and should act in an individual when a gene construct is administered and be in frame with the coding sequence. A promoter of a vector may be constitutive or inducible. It may also contain a replication origin for a replicable expression vector. In addition, it may suitably contain an enhancer, an untranslated region at the 5′ end and 3′ end of a gene of interest, a selection marker (for example, an antibiotic resistance marker), or a replicable unit. A vector may be self-replicating or integrated into a host genomic DNA.

Examples of useful expression regulatory sequences include the early and late promoters of adenovirus, simian virus 40 (SV40), mouse mammary tumor virus (MMTV) promoter, long terminal repeat (LTR) promoter of HIV, Moloney virus, cytomegalovirus (CMV) promoter, Epstein-Barr virus (EBV) promoter, Rous sarcoma virus (RSV) promoter, RNA polymerase II promoter, R-actin promoter, human hemoglobin promoter and human muscle creatine promoter, lac system, trp system, a TAC or TRC system, T3 and T7 promoters, a major operator and promoter region of phage lambda, a regulatory region of the fd code protein, a promoter for phosphoglycerate kinase (PGK) or other glycolytic enzymes, phosphatase promoters, such as Pho5, a promoter of the yeast alpha-mating system, and other constitutive or inducible sequences known to regulate the expression of genes of prokaryotic or eukaryotic cells or their virus and combinations thereof.

In order to increase the expression level of a transgene in a cell, the desired gene and the transcriptional and translational expression regulatory sequences should be operably linked to each other. Generally, “operably linked” means that the linked DNA sequences are in contact, and in the case of a secretory leader, it is in contact and present in a reading frame. For example, if the DNA for a pre-sequence or secretory leader is expressed as a preprotein participating in the secretion of the protein, it can be operably linked to the DNA for the polypeptide, and if a promoter or an enhancer affects the transcription of a sequence, it may be operably linked to the coding sequence, if a ribosome biding site affects the transcription of a sequence, it may be operably linked to the coding sequence, or if a ribosome binding site is placed to promote translation, it can be operably linked to a coding sequence. The linkage of these sequences can be performed by ligation at a convenient restriction site, and in the absence of such site, the linkage can be performed using a synthetic oligonucleotide adapter or linker in accordance with a conventional method.

Those skilled in the art can choose various vectors suitable for the present invention, expression regulatory sequences, a host, etc. in view of the nature of the host cell, the copy number of the vector, the ability to control the copy number, and other proteins encoded by the vector, for example, the expression of an antibiotic marker.

Next, the microorganism is transformed using the recombinant vector.

The term “transformation” means introducing DNA into a host and allowing the DNA to replicate as an extrachromosomal factor or by chromosome integration completion.

A microorganism that can be transformed with the recombinant vector according to the present invention includes both prokaryotic and eukaryotic cells, and a host having high efficiency of introduction of DNA and high efficiency of expression of the introduced DNA can be generally used. Specific examples include, but are not limited to, known eukaryotic and prokaryotic host cells such as the genus Escherichia including Escherichia coli (for example, E. coli DH5a, E. coli JM101, E. coli K12, E. coli W3110, E. coli X1776, E. coli B and E. coli XLI-Blue), the genus Pseudomonas, the genus Bacillus, the genus Streptomyces, the genus Erwinia, the genus Serratia, the genus Providencia, the genus Corynebacterium, the genus Leptospira, the genus Salmonella, the genus Brevibacterium, the genus Hyphomonas, the genus Chromobacterium, the genus Nocardia, fungi or yeast. Once transformed into an appropriate host, the vector can replicate and function independently of the host genome, or, in some cases, integrate into the genome itself.

For the purpose of the present invention, a host cell may be a microorganism having a pathway for biosynthesis of hydroxyacyl-CoA from a carbon source.

Transformation methods include, but are not limited to, using appropriate standard techniques known in the art, such as electroporation, electro injection, microinjection, calcium phosphate co-precipitation, a calcium chloride/rubidium chloride method, a retroviral infection, DEAE-dextran, a cationic liposome method, a polyethylene glycol-mediated uptake, a gene gun, and the like. At this time, a circular vector can be cut with appropriate restriction enzymes and introduced in a linear form.

Next, the above-mentioned transformed microorganism is cultured to produce 4-hydroxybutyrate-2-hydroxybutyrate copolymer.

The transformant expressing the recombinant vector can be cultured in a medium to produce and isolate a large amount of 4-hydroxybutyrate-2-hydroxybutyrate copolymer. The medium and culture conditions can be suitably selected depending on the kind of transformed cells. The conditions such as the temperature, the pH of the medium and the culture time can be appropriately adjusted during culture so as to be suitable for the growth of the cells and the mass production of the copolymer. Examples of such culture methods include, but are not limited to, batch, continuous, and fed-batch culture.

In one embodiment, the culture may be conducted in a medium comprising 2-hydroxybutyrate and/or 4-hydroxybutyrate. Further, if the microorganism is capable of biosynthesizing 2-hydroxybutyrate and 4-hydroxybutyrate from a carbon source such as glucose, the copolymer can be prepared without addition of 2-hydroxybutyrate and/or 4-hydroxybutyrate.

In addition, the culture medium should suitably satisfy the requirements of a particular strain. The medium may include various carbon sources, nitrogen sources, phosphorus, and trace element components. Carbon sources in the medium include sugars and carbohydrates such as glucose, saccharose, lactose, fructose, maltose, starch and cellulose, oils and fats such as soybean oil, sunflower oil, castor oil and coconut oil, fatty acids such as palmitic acid, stearic acid and linoleic acid, alcohol such as glycerol and ethanol, and organic acids such as acetic acid, but are not limited thereto. These materials may be used individually or as a mixture. Examples of the nitrogen source in the medium include peptone, yeast extract, meat extract, malt extract, corn steep liquor, soybean flour and urea or inorganic compounds such as ammonium sulfate, ammonium chloride, ammonium phosphate, ammonium carbonate and ammonium nitrate, but are not limited thereto. The nitrogen source may also be used individually or as a mixture. Examples of phosphorus sources in the medium include, but are not limited to, potassium dihydrogen phosphate or dipotassium hydrogenphosphate or the corresponding sodium-containing salts. In addition, the culture medium may include metal salts such as magnesium sulfate or iron sulfate necessary for growth, or may include, but is not limited to, essential growth materials such as amino acids and vitamins. The above-mentioned raw materials can be added to the culture in a batch or continuous manner by an appropriate method.

In addition, if necessary, basic compounds such as sodium hydroxide, potassium hydroxide, or ammonia, or acidic compounds such as phosphoric acid or sulfuric acid can be used in a suitable manner to adjust the pH of the culture. In addition, bubble formation can be suppressed by using a defoaming agent such as a fatty acid polyglycol ester. Oxygen or oxygen-containing gas (e.g., air) may be injected into the culture to maintain aerobic conditions, and the temperature of the culture may be usually in the range of 20° C. to 45° C., preferably 25° C. to 40° C. The culture may continue until the desired yield of the desired copolymer is obtained.

Next, the produced 4-hydroxybutyrate-2-hydroxybutyrate copolymer is recovered.

The 4-hydroxybutyrate-2-hydroxybutyrate copolymer produced from the recombinant microorganism can be isolated from the cell or culture medium by methods well known in the art. Examples of the method for recovering the 4-hydroxybutyrate-2-hydroxybutyrate copolymer include, but are not limited to, centrifugal method, ultrasonic disruption, filtration, ion exchange chromatography, high performance liquid chromatography (HPLC), and gas chromatography (GC).

### DETAILED DESCRIPTION OF THE EMBODIMENTS

Hereinafter, the present invention will be described in more detail by way of examples. However, the following examples are for illustrative purposes only, and are not intended to limit the present invention thereto.

### Example 1. Preparation of Recombinant Vector for the Preparation of 4-Hydroxybutyrate-2-hydroxybutyrate Copolymer

### 1-1. Preparation of pPs619C1310-CPPCT540 Recombinant Vector

A mutant of propionyl-CoA transferase (CP-PCT) gene derived from Clostridium propionicum was used as the propionyl-CoA transferase gene (pct), and a mutant of PHA synthase gene derived from Pseudomonas sp. MBEL 6-19 (KCTC 11027BP) was used as the PHA synthase gene. The vector used was pBluescript I (Stratagene Co., USA).

First, the entire DNA of Pseudomonas sp. MBEL 6-19 (KCTC 11027BP) was extracted to isolate the PHA synthase (phaC1_{Ps6-19}) gene. Based on the phaCI_{Ps6-19} gene sequence (SEQ ID NO: 3), primers [5′-GAG AGA CAA TCA AAT CAT GAG TAA CAA GAG TAA CG-3′ (SEQ ID NO: 5), 5′-CAC TCA TGC AAG CGT CAC CGT TCG TGC ACG TAC-3′ (SEQ ID NO: 6)] were prepared. PCR was performed using the extracted whole DNA as a template. The obtained PCR product was electrophoresed to identify a gene fragment having a size of 1.7 kb corresponding to the phaC1_{Ps6-19} gene and to obtain a phaC1_{Ps6-19} gene.

In order to express the phaC1_{Ps6-19} synthase, the DNA fragment containing the PHB production operon derived from Ralstonia eutropha H16 was digested from pSYL105 vector (Lee et al., Biotech. Bioeng., 1994, 44: 1337-1347) with BamHI/EcoRI, and inserted into the BamHI/EcoRI recognition site of pBluescript II (Stratagene Co., USA) to prepare a pReCAB recombinant vector. In the pReCAB vector, the PHA synthase (phaC_{RE}) and the monomer feeding enzymes (phaA_{RE} and phaB_{RE}) are constantly expressed by the PHB operon promoter. In order to prepare phaC1_{Ps6-19} synthase gene fragment comprising one BstBI/SbfI recognition site at each end, the endogenous BstBI site was first removed by SDM (site directed mutagenesis) method without amino acid conversion. And then, in order to add BstBI/SbfI recognition site, overlapping PCR was performed using primers [5′-atg ccc gga gcc ggt tcg aa-3′(SEQ ID NO: 7), 5′-CGT TAC TCT TGT TAC TCA TGA TTT GAT TGT CTC TC—3′(SEQ ID NO: 8), 5′-GAG AGA CAA TCA AAT CAT GAG TAA CAA GAG TAA CG-3′ (SEQ ID NO: 9), 5-CAC TCA TGC AAG CGT CAC CGT TCG TGC ACG TAC 3′(SEQ ID NO: 10), 5′-GTA CGT GCA CGA ACG GTG ACG CTT GCA TGA GTG 3′(SEQ ID NO: 11), 5′-aac ggg agg gaa cct gca gg-3′(SEQ ID NO: 12)]. The recombinant vector pPs619C1-ReAB was prepared by cleaving the pReCAB vector with BstBI/SbfI to remove the R. eutropha H16 PHA synthase (phaC_{RE}), and then inserting the phaC1_{Ps6-19} gene obtained above into the BstBI/SbfI recognition site.

Three amino acid positions affecting short chain length (SCL) activity were found by sequencing of amino acid sequences. pPs619C1300-ReAB containing phaC1_{Ps6-19}300 which is a phaC1_{Ps6-19} synthase mutant containing E130D, S325T and Q481M was prepared using SDM method with primers [5′-CTG ACC TTG CTG GTG ACC GTG CTT GAT ACC ACC-3′ (SEQ ID NO: 13), 5-GGT GGT ATC AAG CAC GGT CAC CAG CAA GGT CAG-3′(SEQ ID NO: 14), 5′-CGA GCA GCG GGC ATA TC A TGA GCA TCC TGA ACC CGC-3′(SEQ ID NO: 15), 5′-GCG GGT TCA GGA TGC TCA TGA TAT GCC CGC TGC TCG-3′(SEQ ID NO: 16), 5′-atc aac ctc atg acc gat gcg atg gcg ccg acc-3′ (SEQ ID NO: 17), 5′-ggt cgg cgc cat cgc atc ggt cat gag gtt gat-3′ (SEQ ID NO: 18)].

Here, in order to construct a constitutively expressed system in the operon form in which the propionyl-CoA transferase is coexpressed, a propyl-CoA transferase (CP-PCT) derived from Clostridium propionicum was used. A fragment obtained by PCR using the chromosomal DNA of Clostridium propionicum and primers [5′-GGAATTCATGAGAAAGGTTCCCATTATTACCGCAGATGA-3′(SEQ ID NO: 19), 5′-gc tctaga tta gga ctt cat ttc ctt cag acc cat taa gcc ttc tg-3′(SEQ ID NO: 20)] was used as CP-PCT. At this time, the NdeI site existing in the wild-type CP-PCT was removed using the SDM method for easy cloning. Further, in order to add the SbfI/NdeI recognition site, overlapping PCR was performed using primers [5′-agg cct gca ggc gga taa caa ttt cac aca gg-3′ (SEQ ID NO: 21), 5′-gcc cat atg tct aga tta gga ctt cat ttc c-3′ (SEQ ID NO: 22)]. The pPs619C1300-CPPCT vector was prepared by cleaving the pPs619C1300-ReAB vector with SbfI/NdeI to remove the monomer feeding enzymes (phaA_{RE} and phaB_{RE}) derived from Ralstonia eutrophus H16 and then inserting the PCR-cloned CP-PCT gene into the SbfI/NdeI recognition site.

Next, error-prone PCR was performed under the condition that Mn²⁺ was added and dNTPs were present in different concentrations, using the above prepared pPs619C1300-CPPCT as a template and primers [5′-CGCCGGCAGGCCTGCAGG-3′(SEQ ID NO: 23), 5′-GGCAGGTCAGCCCATATGTC-3′(SEQ ID NO: 24)] to introduce a random mutation into the CP-PCT gene. Thereafter, PCR was performed under normal conditions using the above primers to amplify the PCR fragment containing a random mutation. The pPs619C1300-CPPCT vector was digested with SbfI/NdeI to remove wild-type CP-PCT, and then the ligation mixture in which the amplified mutant PCR fragment was inserted into the SbfI/NdeI recognition site was prepared and introduced into E. coli JM109 to obtain 10⁵ sized CP-PCT library. The prepared CP-PCT library was grown in a polymer detection medium (LB agar, glucose 20 g/L, 3HB Ig/L, Nile red 0.5 μg/ml) for 3 days and then subjected to a screening to identify a polymer production and about 80 candidates were selected first. These candidates were subjected to liquid culture (LB agar, glucose 20 g/L, 3HB 1 g/L, ampicillin 100 mg/L, 37° C.) for 4 days under the conditions of polymer production and 2 variants, CP-PCT Variant 512 (comprising nucleic acid substitution A1200G) and CP-PCT Variant 522 (comprising nucleic acid substitutions T78C, T669C, A1125G and T1158C) were selected by FACS (Florescence Activated Cell Sorting) analysis. Various CP-PCT variants were obtained by random mutagenesis using the above-mentioned error-prone PCR method based on the above-selected primary mutants (CP-PCT Variant 512 and CP-PCT Variant 522). The CP-PCT Variant 540 (comprising Val193Ala and silent mutations T78C, T669C, A1125G, and T1158C) was secondly selected among them to prepare pPs619C1300-CPPCT540 vector.

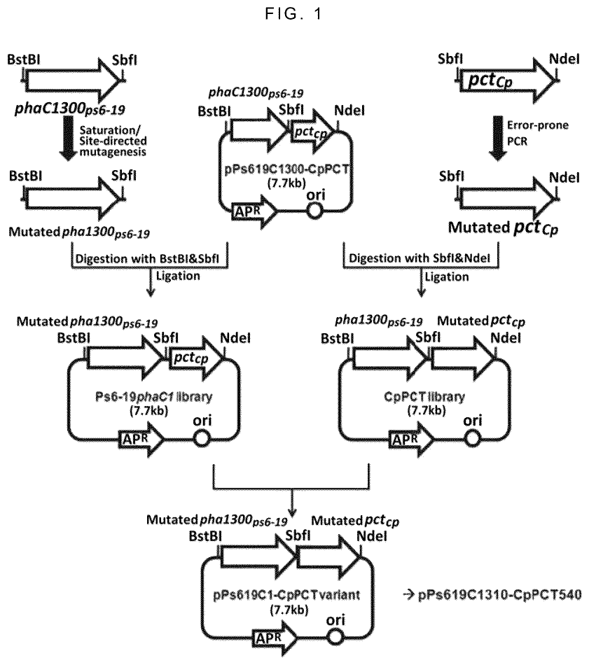

Further, pPs619C1310-CPPCT540 vector containing the PHA synthase variant (phaC1_{Ps6-19}310) derived from Pseudomonas sp. MBEL 6-19 having the amino acid sequence with mutations of E130D, S477F and Q481K was prepared using the SDM method using the primers [5′-gaa ttc gtg ctg tcg agc cgc ggg cat atc-3′ (SEQ ID NO: 25), 5′-gat atg ccc gcg gct cga cag cac gaa ttc-3′(SEQ ID NO: 26), 5′-ggg cat atc aag agc atc ctg aac ccg c-3′(SEQ ID NO: 27), 5′-g cgg gtt cag gat gct ctt gat atg ccc-3′(SEQ ID NO: 28)] based on the above prepared phaC1_{Ps6-19} synthase mutant (phaC1_{Ps6-19}300) (FIG. 1).

### 1-2. Preparation of PPs619C1249.18H-CPPCT540 Recombinant Vector

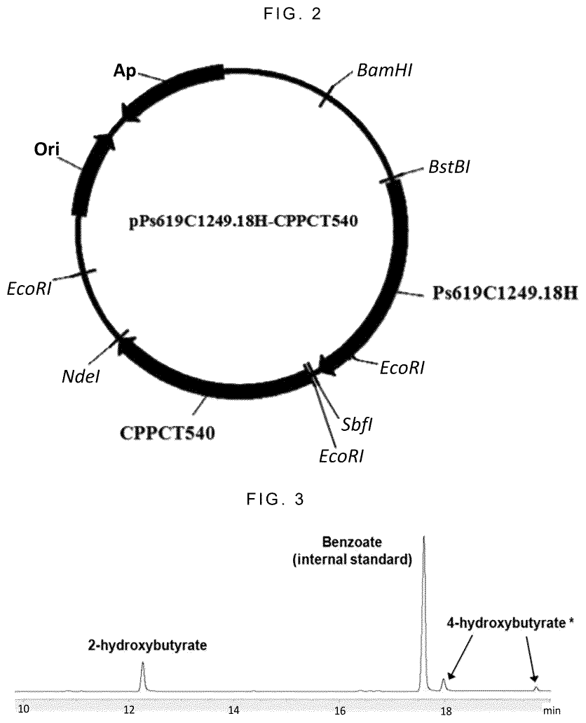

Error-prone PCR was performed using the primers [5′-ATGCCCGGAGCCGGTTCGAA-3′(SEQ ID NO: 29) and 5′-GAAATTGTTATCCGCCTGCAGG-3′(SEQ ID NO: 30)] and the pPs619C1310-CPPCT540 vector prepared in 1-1 above as a template. After performing error-prone PCR, PCR was performed again using the above primers to amplify the PCR fragment containing the mutation, and the amplified mutants were inserted into the BstBI/SbfI site of the pPs619C1310-CPPCT540 vector to construct a library of mutants. The prepared mutant library was transformed into E. coli XL-1 Blue and the transformants were cultured in PHB detection medium (LB agar, glucose 20 g/L, Nile red 0.5 μg/ml) for 3 days. The finally selected variant through screening after culture was pPs619C1249.18H having the amino acid mutations of L18H, V24A, K91R, M128V, E130D, N246S, S325T, S477G, Q481K and A527S. Thus, the PPs619C1249.18H-CPPCT540 vector was prepared (FIG. 2).

### Example 2. Preparation of E. coli XL1-Blue Variant with IdhA Gene Knock-Out

In order to produce a lactate-free polymer based on Escherichia coli XL1-Blue (Stratagene, USA), D-lactate dehydrogenase (LdhA) involved in lactate production during the metabolism of E. coli was knocked out in genomic DNA. Genetic deletions were made using the well-known red-recombination method. The oligomers used to delete IdhA were synthesized to have the sequences of SEQ ID NO: 31(5′-atcagcgtacccgtgatgctaacttctctctggaaggtctgaccggctttaattaaccctcactaa agggcg-3′) and SEQ ID NO: 32 (5′-atcagcgtacccgtgatgctaacttctctctggaaggtctgaccggctttaattaaccctcactaa agggcg-3′).

### Example 3. Preparation of 4-hydroxybutyrate-2-Hydroxybutyrate Copolymer

The recombinant vector prepared in Example 1 was transformed into E. coli XLI-BlueΔldhA with IdhA gene knock out prepared in Example 2 using electroporation, thereby obtaining recombinant E. coli XLI-BlueΔldhA. The flask culture was carried out to prepare the above-mentioned terpolymer using the recombinant E. coli. First, for the seed culture, the recombinant E. coli was cultured in 3 mL of LB medium [Bacto™ Triptone (BD) 10 g/L, Bacto™ yeast extract (BD) 5 g/L, NaCL (amresco) 10 g/L] containing 100 mg/L ampicillin and 20 mg/L kanamycin for 12 hours. For the main culture, 1 ml of the seed culture was inoculated into 100 ml MR medium(Glucose 10 g, KH₂PO₄ 6.67 g, (NH₄)₂HPO₄ 4 g, MgSO₄·7H₂O 0.8 g, citric acid 0.8 g, and trace metal solution 5 mL per 1 L; herein, trace metal solution contains 5 M HCl 5 mL, FeSO₄·7H₂O 10 g, CaCl₂) 2 g, ZnSO₄·7H₂O 2.2 g, MnSO₄·4H₂O 0.5 g, CuSO₄·5H₂O 1 g, (NH₄)6 Mo₇O₂·4H₂O 0.1 g, and Na₂B₄O₂·10H₂O 0.02 g per 1 L) supplemented with 1 g/L 4-hydroxybutyrate(4-HB), 1 g/L 2-hydroxybutyrate(2-HB), 100 mg/L ampicillin, 20 mg/L kanamycin, and 10 mg/L thiamine, and cultured at 30° C. for 3 days with stirring at 250 rpm.

The culture solution was centrifuged at 4,000 rpm at 4° C. for 10 minutes to recover the cells. The recovered cells were washed twice with distilled water and dried at 80° C. for 12 hours. After quantification of the cells, the cells were reacted with methanol under a sulfuric acid catalyst using chloroform as a solvent at 100° C. Distilled water having a volume corresponding to half of chloroform was added thereto at room temperature, and the mixture was allowed to stand until it was separated into two layers. Among the two layers, the chloroform layer in which the monomers of the methylated polymer were dissolved was collected, and the components of the polymer were analyzed by gas chromatography (GC). Benzoate was used as an internal standard material. The GC analysis conditions used are shown in Table 1 below.

As shown in Table 2 and FIG. 3, the result of GC analysis showed that 4-hydroxybutyrate-2-hydroxybutyrate copolymer was produced by the recombinant E. coli.

TABLE 1
GC Analysis conditions

| Item | Quality | |
|---|---|---|
| Model | Hewlett Packard 6890N | |
| Detector | Flame ionization detector (FID) | |
| Column | Alltech Capillary AT ™-WAX, | |
| | 30 | m, |
| | 0.53 | mm |
| Liquid phase | 100% polyethylene Glycol | |
| Inj. port temp/Det. port temp | 250° | C./250° C. |
| Carrier gas | He | |
| Total flow | 3 | ml/min |
| septum purge went flow | 1 | ml/min |
| Column head pressure | 29 | kPa |
| Injection port mode | Splitless | |
| Injection volumn/Solvent | 1 | μl/chloroform |
| Initial temp./Time | 80° | C./5 min |
| Final temp./Time | 230° | C./5 min |
| Ramp of temp. | 7.5° | C./min |

TABLE 2

| Total PHA | Polymer (mol %) | |
|---|---|---|
| content(wt %) | 4HB | 2HB |
| 5.93 | 40.3 | 59.7 |

### Example 4. Analysis of Physical Properties in Accordance with the Molar Ratio of Each Monomer in 4-Hydroxybutyrate-2-Hydroxybutyrate Copolymer

In the method described in Example 3, the concentrations of 4-hydroxybutyrate and 2-hydroxybutyrate in the main culture medium were varied from 0 to 3 g/L during culture for production of 4-hydroxybutyrate-2-hydroxybutyrate copolymer. After culturing, only the cells were recovered from the culture solution by centrifugation for polymer purification, and washed twice with distilled water and then freeze-dried. Next, chloroform was added to the lyophilized cells to a concentration of about 30 g/L based on the polymer concentration, and the polymer was extracted at room temperature for 24 hours with stirring using a magnetic stirrer. Then, chloroform, distilled water, and methanol were added at a ratio of 2:1:1, and the resultant mixture was subjected to layer separation at room temperature. Then, a polymer extract solution at bottom layer was separated using a separating funnel. Then, the cell residues were separated and removed using a filter paper. Next, almost all of the chloroform was removed from the filtered polymer solution through evaporation, and then methanol was added to precipitate the polymer. The precipitated polymer was collected by centrifugation and finally dried in a dry oven (75° C.).

The molar ratio of monomers in the polymer was confirmed by GC analysis described in Example 3 (Table 3). In Table 3 below, the polymer contents (wt %) are the PHA polymer contents relative to the dry cell weight.

TABLE 3

| 2HB | 4HB | 2HB | 4HB | |
|---|---|---|---|---|
| concentration | concentration | in the | in the | Polymer |
| in the | in the | polymer | polymer | contents |
| medium (g/L) | medium (g/L) | (mol %) | (mol %) | (wt %) |
| 3.0 | 0.0 | 100 ± 0.0 | 0.0 ± 0.0 | 13.0 ± 4.1 |
| 2.7 | 0.3 | 79.0 ± 1.1 | 21.0 ± 1.1 | 40.3 ± 2.8 |
| 2.4 | 0.6 | 70.0 ± 1.6 | 30.0 ± 1.6 | 54.2 ± 3.3 |
| 2.1 | 0.9 | 58.2 ± 1.4 | 41.8 ± 1.4 | 54.7 ± 5.0 |
| 1.8 | 1.2 | 53.7 ± 0.9 | 46.3 ± 0.9 | 56.2 ± 2.8 |
| 1.5 | 1.5 | 41.2 ± 5.0 | 58.8 ± 5.0 | 54.0 ± 2.6 |
| 1.2 | 1.8 | 33.7 ± 4.4 | 66.3 ± 4.4 | 51.9 ± 4.9 |
| 0.9 | 2.1 | 26.6 ± 0.2 | 73.4 ± 0.2 | 57.3 ± 0.4 |
| 0.6 | 2.4 | 17.3 ± 0.3 | 82.7 ± 0.3 | 51.3 ± 1.8 |
| 0.3 | 2.7 | 9.4 ± 0.9 | 90.6 ± 0.9 | 49.6 ± 4.4 |
| 0.0 | 3.0 | 0.0 ± 0.0 | 100.0 ± 0.0 | 53.7 ± 2.7 |

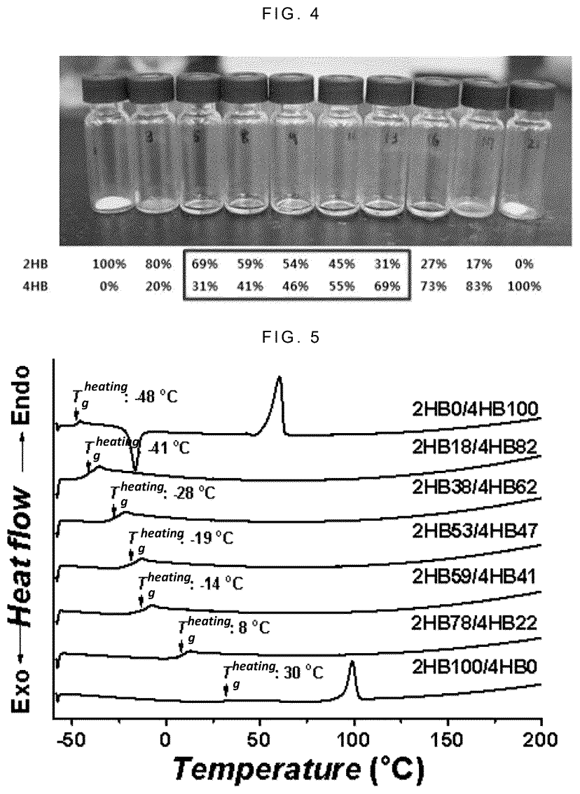

As a result of analysis of physical properties of some samples, it was found that when the molar ratios of 2HB and 4HB monomers were 30% or more, respectively, the copolymer had enough adhesive property to be used as a bioadhesive and was present as a liquid at room temperature (FIG. 4).

Differential scanning calorimetry (DSC) analysis was also performed to investigate the thermal properties of polymers according to the molar ratios of monomers. Heating was performed twice during DSC measurement, and the glass transition temperature (Tg) and the melting temperature (Tm) were measured at the second heating. The initial temperature at the time of heating was −60° C., the rate of temperature rise was 10° C./min, and the final temperature was 200° C. Nitrogen was used as a carrier gas for the DSC analysis. As a result of the DSC analysis, crystallization was observed for homopolymers of 2HB and 4HB, but crystallization was not observed for copolymers of 2HB and 4HB, indicating that the copolymer is amorphous polymer. In addition, the melting temperature (Tm) was not observed in the copolymer of 2HB and 4′HB, and it was confirmed that the glass transition temperature (Tg) was increased with increasing the molar ratio of 2HB.

1-8. (canceled)

9. A method for preparing a copolymer comprising 4-hydroxybutyrate and 2-hydroxybutyrate as a repeating unit, comprising
culturing a microorganism which has a weakened or deficient activity of lactate dehydrogenase and comprises a gene encoding an enzyme converting 2-hydroxyalkanoate into 2-hydroxyalkanoyl-CoA and converting 4-hydroxyalkanoate into 4-hydroxyalkanoyl-CoA and a gene encoding a polyhydroxyalkanoate synthase using 2-hydroxyalkanoyl-CoA and 4-hydroxyalkanoyl-CoA as a substrate.

10. The method according to claim 9, wherein the microorganism is obtained by transforming a microorganism with a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate.

11. The method according to claim 9, wherein the enzyme converting 2-hydroxyalkanoate into 2-hydroxyalkanoyl-CoA and converting 4-hydroxyalkanoate into 4-hydroxyalkanoyl-CoA is propionyl-CoA transferase.

12. The method according to claim 9, wherein the gene encoding for the enzyme converting 2-hydroxyalkanoate into 2-hydroxyalkanoyl-CoA and converting 4-hydroxyalkanoate into 4-hydroxyalkanoyl-CoA consists of a nucleotide sequence selected from the group consisting of:
(a) a nucleotide sequence of SEQ ID NO: 1;
(b) a nucleotide sequence of SEQ ID NO: 1 comprising A1200G mutation;
(c) a nucleotide sequence of SEQ ID NO: 1 comprising T78C, T669C, A1125G and T1158C mutation;
(d) a nucleotide sequence of SEQ ID NO: 1 comprising A1200G mutation and a mutation resulting in Gly335Asp mutation in the amino acid sequence corresponding to SEQ ID NO: 1;
(e) a nucleotide sequence of SEQ ID NO: 1 comprising A1200G mutation and a mutation resulting in Ala243Thr mutation in the amino acid sequence corresponding to SEQ ID NO: 1;
(f) a nucleotide sequence of SEQ ID NO: 1 comprising T669C, A1125G and T1158C mutations and a mutation resulting in Asp65Gly mutation in the amino acid sequence corresponding to SEQ ID NO: 1;
(g) a nucleotide sequence of SEQ ID NO: 1 comprising A1200G mutation and a mutation resulting in Asp257Asn mutation in the amino acid sequence corresponding to SEQ ID NO: 1;
(h) a nucleotide sequence of SEQ ID NO: 1 comprising T669C, A1125G and T1158C mutations and a mutation resulting in Asp65Asn mutation in the amino acid sequence corresponding to SEQ ID NO: 1;
(i) a nucleotide sequence of SEQ ID NO: 1 comprising T669C, A1125G and Ti 158C mutations and a mutation resulting in Thr199Ile mutation in the amino acid sequence corresponding to SEQ ID NO: 1; and
(j) a nucleotide sequence of SEQ ID NO: 1 comprising T78C, T669C, A1125G and T1158C mutations and a mutation resulting in Val93Ala mutation in the amino acid sequence corresponding to SEQ ID NO: 1.

13. The method according to claim 9, wherein the polyhydroxyalkanoate synthase is polyhydroxyalkanoate synthase derived from Pseudomonas sp.6-19.

14. The method according to claim 9, wherein the gene encoding polyhydroxyalkanoate synthase consists of:
a nucleotide sequence corresponding to an amino acid sequence of SEQ ID NO: 4 or
an amino acid sequence of SEQ ID NO: 4 comprising at least one mutation selected from the group consisting of L18H, V24A, K91R, M128V, E130D, N246S, S325T, S477R, S477H, S477F, S477Y, S477G, Q481M, Q481K, Q481R, and A527S.

15. The method according to claim 9, wherein the gene encoding polyhydroxyalkanoate synthase consists of a nucleotide sequence corresponding to an amino acid sequence of SEQ ID NO: 4 comprising a mutation selected from the group consisting of:
(i) S325T and Q481M;
(ii) E130D, S325T and Q481M;
(iii) E130D, S325T, S477R and Q481M;
(iv) E130D, S477F and Q481K; and
(v) L18H, V24A, K91R, M128V, E130D, N246S, S325T, S477G, Q481K and A527S.

16. The method according to claim 9, wherein the culture is performed in a medium comprising 2-hydroxybutyrate and 4-hydroxybutyrate.

17. A microorganism producing a copolymer comprising 2-hydroxybutyrate and 4-hydroxybutyrate as a repeating unit, wherein the microorganism has a weakened or deficient activity of lactate dehydrogenase and comprises a gene encoding an enzyme converting 2-hydroxyalkanoate into 2-hydroxyalkanoyl-CoA and converting 4-hydroxyalkanoate into 4-hydroxyalkanoyl-CoA and a gene encoding a PHA synthase using 2-hydroxyalkanoyl-CoA and 4-hydroxyalkanoyl-CoA as a substrate.

18. The microorganism according to claim 17, wherein the enzyme converting 2-hydroxyalkanoate into 2-hydroxyalkanoyl-CoA and converting 4-hydroxyalkanoate into 4-hydroxyalkanoyl-CoA is propionyl-CoA transferase.

19. The microorganism according to claim 17, wherein the gene encoding the enzyme converting 2-hydroxyalkanoate into 2-hydroxyalkanoyl-CoA and converting 4-hydroxyalkanoate into 4-hydroxyalkanoyl-CoA consists of a nucleotide sequence selected from the group consisting of:
(a) a nucleotide sequence of SEQ ID NO: 1;
(b) a nucleotide sequence of SEQ ID NO: 1 comprising A1200G mutation;
(c) a nucleotide sequence of SEQ ID NO: 1 comprising T78C, T669C, A1125G and T1158C mutation;
(d) a nucleotide sequence of SEQ ID NO: 1 comprising A1200G mutation and a mutation resulting in Gly335Asp mutation in the amino acid sequence corresponding to SEQ ID NO: 1;
(e) a nucleotide sequence of SEQ ID NO: 1 comprising A1200G mutation and a mutation resulting in Ala243Thr mutation in the amino acid sequence corresponding to SEQ ID NO: 1;
(f) a nucleotide sequence of SEQ ID NO: 1 comprising T669C, A1125G and T1158C mutations and a mutation resulting in Asp65Gly mutation in the amino acid sequence corresponding to SEQ ID NO: 1;
(g) a nucleotide sequence of SEQ ID NO: 1 comprising A1200G mutation and a mutation resulting in Asp257Asn mutation in the amino acid sequence corresponding to SEQ ID NO: 1;
(h) a nucleotide sequence of SEQ ID NO: 1 comprising T669C, A1125G and T1158C mutations and a mutation resulting in Asp65Asn mutation in the amino acid sequence corresponding to SEQ ID NO: 1;
(i) a nucleotide sequence of SEQ ID NO: 1 comprising T669C, A1125G and Ti 158C mutations and a mutation resulting in Thr199Ile mutation in the amino acid sequence corresponding to SEQ ID NO: 1; and
(j) a nucleotide sequence of SEQ ID NO: 1 comprising T78C, T669C, A1125G and T1158C mutations and a mutation resulting in Val93Ala mutation in the amino acid sequence corresponding to SEQ ID NO: 1.

20. The microorganism according to claim 17,
wherein the polyhydroxyalkanoate synthase is polyhydroxyalkanoate synthase derived from Pseudomonas sp.6-19.

21. The microorganism according to claim 17, wherein the gene encoding polyhydroxyalkanoate synthase consists of:
a nucleotide sequence corresponding to an amino acid sequence of SEQ ID NO: 4 or
an amino acid sequence of SEQ ID NO: 4 comprising at least one mutation selected from the group consisting of L18H, V24A, K91R, M128V, E130D, N246S, S325T, S477R, S477H, S477F, S477Y, S477G, Q481M, Q481K, Q481R and A527S.

22. The microorganism according to claim 17, wherein the gene encoding polyhydroxyalkanoate synthase consists of a nucleotide sequence corresponding to an amino acid sequence of SEQ ID NO: 4 comprising a mutation selected from the group consisting of
(i) S325T and Q481M;
(ii) E130D, S325T and Q481M;
(iii) E130D, S325T, S477R and Q481M;
(iv) E130D, S477F and Q481K; and
(v) L18H, V24A, K91R, M128V, E130D, N246S, S325T, S477G, Q481K and A527S.


SEQUENCE LISTING (32 sequences)

SEQ ID NO: 1 - DNA, 1575 bp, Clostridium propionicum
  gene 1..1575: note = propionyl-CoA transferase
  source 1..1575: mol_type = genomic DNA
atgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacctatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcatgtagctcttattcgtggtacttacgctgatgaaagcggaaatatcacatttgagaaagaagttgctcctctggaaggaacttcagtatgccaggctgttaaaaacagtggcggtatcgttgtagttcaggttgaaagagtagtaaaagctggtactcttgaccctcgtcatgtaaaagttccaggaatttatgttgactatgttgttgttgctgacccagaagatcatcagcaatctttagattgtgaatatgatcctgcattatcaggcgagcatagaagacctgaagttgttggagaaccacttcctttgagtgcaaagaaagttattggtcgtcgtggtgccattgaattagaaaaagatgttgctgtaaatttaggtgttggtgcgcctgaatatgtagcaagtgttgctgatgaagaaggtatcgttgattttatgactttaactgctgaaagtggtgctattggtggtgttcctgctggtggcgttcgctttggtgcttcttataatgcggatgcattgatcgatcaaggttatcaattcgattactatgatggcggcggcttagacctttgctatttaggcttagctgaatgcgatgaaaaaggcaatatcaacgtttcaagatttggccctcgtatcgctggttgtggtggtttcatcaacattacacagaatacacctaaggtattcttctgtggtactttcacagcaggtggcttaaaggttaaaattgaagatggcaaggttattattgttcaagaaggcaagcagaaaaaattcttgaaagctgttgagcagattacattcaatggtgacgttgcacttgctaataagcaacaagtaacttatattacagaaagatgcgtattccttttgaaggaagatggtttgcacttatctgaaattgcacctggtattgatttgcagacacagattcttgacgttatggattttgcacctattattgacagagatgcaaacggccaaatcaaattgatggacgctgctttgtttgcagaaggcttaatgggtctgaaggaaatgaagtcctaa

SEQ ID NO: 2 - protein, 524 aa, Clostridium propionicum
  PEPTIDE 1..524: note = propionyl-CoA transferase
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 3 - DNA, 1677 bp, Pseudomonas sp. 6-19
  gene 1..1677: note = PHA synthase
  source 1..1677: mol_type = genomic DNA
atgagtaacaagagtaacgatgagttgaagtatcaagcctctgaaaacaccttggggcttaatcctgtcgttgggctgcgtggaaaggatctactggcttctgctcgaatggtgcttaggcaggccatcaagcaaccggtgcacagcgtcaaacatgtcgcgcactttggtcttgaactcaagaacgtactgctgggtaaatccgggctgcaaccgaccagcgatgaccgtcgcttcgccgatccggcctggagccagaacccgctctataaacgttatttgcaaacctacctggcgtggcgcaaggaactccacgactggatcgatgaaagtaacctcgcccccaaggatgtggcgcgtgggcacttcgtgatcaacctcatgaccgaagcgatggcgccgaccaacaccgcggccaacccggcggcagtcaaacgcttttttgaaaccggtggcaaaagcctgctcgacggcctctcgcacctggccaaggatctggtacacaacggcggcatgccgagccaggtcaacatgggtgcattcgaggtcggcaagagcctgggcgtgaccgaaggcgcggtggtgtttcgcaacgatgtgctggaactgatccagtacaagccgaccaccgagcaggtatacgaacgcccgctgctggtggtgccgccgcagatcaacaagttctacgttttcgacctgagcccggacaagagcctggcgcggttctgcctgcgcaacaacgtgcaaacgttcatcgtcagctggcgaaatcccaccaaggaacagcgagagtggggcctgtcgacctacatcgaagccctcaaggaagcggttgacgtcgttaccgcgatcaccggcagcaaagacgtgaacatgctcggggcctgctccggcggcatcacttgcactgcgctgctgggccattacgcggcgattggcgaaaacaaggtcaacgccctgaccttgctggtgagcgtgcttgataccaccctcgacagcgacgtcgccctgttcgtcaatgaacagacccttgaagccgccaagcgccactcgtaccaggccggcgtactggaaggccgcgacatggcgaaggtcttcgcctggatgcgccccaacgatctgatctggaactactgggtcaacaattacctgctaggcaacgaaccgccggtgttcgacatcctgttctggaacaacgacaccacacggttgcccgcggcgttccacggcgacctgatcgaactgttcaaaaataacccactgattcgcccgaatgcactggaagtgtgcggcacccccatcgacctcaagcaggtgacggccgacatcttttccctggccggcaccaacgaccacatcaccccgtggaagtcctgctacaagtcggcgcaactgtttggcggcaacgttgaattcgtgctgtcgagcagcgggcatatccagagcatcctgaacccgccgggcaatccgaaatcgcgctacatgaccagcaccgaagtggcggaaaatgccgatgaatggcaagcgaatgccaccaagcatacagattcctggtggctgcactggcaggcctggcaggcccaacgctcgggcgagctgaaaaagtccccgacaaaactgggcagcaaggcgtatccggcaggtgaagcggcgccaggcacgtacgtgcacgaacgg

SEQ ID NO: 4 - protein, 559 aa, Pseudomonas sp. 6-19
  PEPTIDE 1..559: note = PHA synthase
MSNKSNDELKYQASENTLGLNPVVGLRGKDLLASARMVLRQAIKQPVHSVKHVAHFGLELKNVLLGKSGLQPTSDDRRFADPAWSQNPLYKRYLQTYLAWRKELHDWIDESNLAPKDVARGHFVINLMTEAMAPTNTAANPAAVKRFFETGGKSLLDGLSHLAKDLVHNGGMPSQVNMGAFEVGKSLGVTEGAVVFRNDVLELIQYKPTTEQVYERPLLVVPPQINKFYVFDLSPDKSLARFCLRNNVQTFIVSWRNPTKEQREWGLSTYIEALKEAVDVVTAITGSKDVNMLGACSGGITCTALLGHYAAIGENKVNALTLLVSVLDTTLDSDVALFVNEQTLEAAKRHSYQAGVLEGRDMAKVFAWMRPNDLIWNYWVNNYLLGNEPPVFDILFWNNDTTRLPAAFHGDLIELFKNNPLIRPNALEVCGTPIDLKQVTADIFSLAGTNDHITPWKSCYKSAQLFGGNVEFVLSSSGHIQSILNPPGNPKSRYMTSTEVAENADEWQANATKHTDSWWLHWQAWQAQRSGELKKSPTKLGSKAYPAGEAAPGTYVHER

SEQ ID NO: 5 - DNA, 35 bp, synthetic construct
  misc_feature 1..35: note = primer
  source 1..35: mol_type = other DNA
gagagacaatcaaatcatgagtaacaagagtaacg

SEQ ID NO: 6 - DNA, 33 bp, synthetic construct
  misc_feature 1..33: note = primer
  source 1..33: mol_type = other DNA
cactcatgcaagcgtcaccgttcgtgcacgtac

SEQ ID NO: 7 - DNA, 20 bp, synthetic construct
  misc_feature 1..20: note = primer
  source 1..20: mol_type = other DNA
atgcccggagccggttcgaa

SEQ ID NO: 8 - DNA, 35 bp, synthetic construct
  misc_feature 1..35: note = primer
  source 1..35: mol_type = other DNA
cgttactcttgttactcatgatttgattgtctctc

SEQ ID NO: 9 - DNA, 35 bp, synthetic construct; identical to SEQ ID NO: 5
  misc_feature 1..35: note = primer
  source 1..35: mol_type = other DNA

SEQ ID NO: 10 - DNA, 33 bp, synthetic construct; identical to SEQ ID NO: 6
  misc_feature 1..33: note = primer
  source 1..33: mol_type = other DNA

SEQ ID NO: 11 - DNA, 33 bp, synthetic construct
  misc_feature 1..33: note = primer
  source 1..33: mol_type = other DNA
gtacgtgcacgaacggtgacgcttgcatgagtg

SEQ ID NO: 12 - DNA, 20 bp, synthetic construct
  misc_feature 1..20: note = primer
  source 1..20: mol_type = other DNA
aacgggagggaacctgcagg

SEQ ID NO: 13 - DNA, 33 bp, synthetic construct
  misc_feature 1..33: note = primer
  source 1..33: mol_type = other DNA
ctgaccttgctggtgaccgtgcttgataccacc

SEQ ID NO: 14 - DNA, 33 bp, synthetic construct
  misc_feature 1..33: note = primer
  source 1..33: mol_type = other DNA
ggtggtatcaagcacggtcaccagcaaggtcag

SEQ ID NO: 15 - DNA, 36 bp, synthetic construct
  misc_feature 1..36: note = primer
  source 1..36: mol_type = other DNA
cgagcagcgggcatatcatgagcatcctgaacccgc

SEQ ID NO: 16 - DNA, 36 bp, synthetic construct
  misc_feature 1..36: note = primer
  source 1..36: mol_type = other DNA
gcgggttcaggatgctcatgatatgcccgctgctcg

SEQ ID NO: 17 - DNA, 33 bp, synthetic construct
  misc_feature 1..33: note = primer
  source 1..33: mol_type = other DNA
atcaacctcatgaccgatgcgatggcgccgacc

SEQ ID NO: 18 - DNA, 33 bp, synthetic construct
  misc_feature 1..33: note = primer
  source 1..33: mol_type = other DNA
ggtcggcgccatcgcatcggtcatgaggttgat

SEQ ID NO: 19 - DNA, 39 bp, synthetic construct
  misc_feature 1..39: note = primer
  source 1..39: mol_type = other DNA
ggaattcatgagaaaggttcccattattaccgcagatga

SEQ ID NO: 20 - DNA, 46 bp, synthetic construct
  misc_feature 1..46: note = primer
  source 1..46: mol_type = other DNA
gctctagattaggacttcatttccttcagacccattaagccttctg

SEQ ID NO: 21 - DNA, 32 bp, synthetic construct
  misc_feature 1..32: note = primer
  source 1..32: mol_type = other DNA
aggcctgcaggcggataacaatttcacacagg

SEQ ID NO: 22 - DNA, 31 bp, synthetic construct
  misc_feature 1..31: note = primer
  source 1..31: mol_type = other DNA
gcccatatgtctagattaggacttcatttcc

SEQ ID NO: 23 - DNA, 18 bp, synthetic construct
  misc_feature 1..18: note = primer
  source 1..18: mol_type = other DNA
cgccggcaggcctgcagg

SEQ ID NO: 24 - DNA, 20 bp, synthetic construct
  misc_feature 1..20: note = primer
  source 1..20: mol_type = other DNA
ggcaggtcagcccatatgtc

SEQ ID NO: 25 - DNA, 30 bp, synthetic construct
  misc_feature 1..30: note = primer
  source 1..30: mol_type = other DNA
gaattcgtgctgtcgagccgcgggcatatc

SEQ ID NO: 26 - DNA, 30 bp, synthetic construct
  misc_feature 1..30: note = primer
  source 1..30: mol_type = other DNA
gatatgcccgcggctcgacagcacgaattc

SEQ ID NO: 27 - DNA, 28 bp, synthetic construct
  misc_feature 1..28: note = primer
  source 1..28: mol_type = other DNA
gggcatatcaagagcatcctgaacccgc

SEQ ID NO: 28 - DNA, 28 bp, synthetic construct
  misc_feature 1..28: note = primer
  source 1..28: mol_type = other DNA
gcgggttcaggatgctcttgatatgccc

SEQ ID NO: 29 - DNA, 20 bp, synthetic construct
  misc_feature 1..20: note = primer
  source 1..20: mol_type = other DNA
atgcccggagccggttcgaa

SEQ ID NO: 30 - DNA, 22 bp, synthetic construct
  misc_feature 1..22: note = primer
  source 1..22: mol_type = other DNA
gaaattgttatccgcctgcagg

SEQ ID NO: 31 - DNA, 72 bp, synthetic construct
  misc_feature 1..72: note = oligomer
  source 1..72: mol_type = other DNA
atcagcgtacccgtgatgctaacttctctctggaaggtctgaccggctttaattaaccctcactaaagggcg

SEQ ID NO: 32 - DNA, 72 bp, synthetic construct; identical to SEQ ID NO: 31
  misc_feature 1..72: note = oligomer
  source 1..72: mol_type = other DNA